(19) United States
(12) Patent Application Publication
(10) Pub. No.: US 2004/0235130 A1
(43) Pub. Date: Nov. 25, 2004

(54) Oxygen-absorbing agent in the form of a pourable granulate

(76) Inventor: Rainer Brandsch, Gilching (DE)

Correspondence Address: Scott R Cox, Lynch Cox Gilman & Mahan, Suite 2200, 400 West Market Street, Louisville, KY, 40202, US

(21) Appl. No.: 10/489,575

(22) PCT Filed: Aug. 27, 2002
(86) PCT No.: PCT/EP02/09560

(30) Foreign Application Priority Data
Sep. 25, 2001 (DE) 101 47 209.9

(51) Int. Cl. C12N 9/04; A61K 33/00

(52) U.S. Cl. 435/190; 424/600; 424/94.4

(57) ABSTRACT

The invention concerns a free-flowing, oxygen-absorbing granulate, comprising:

a) a core region, containing an substrate for an oxygen-removing enzyme, as well as optionally at least one binder,

b) a shell, containing an oxygen-removing enzyme or enzyme mixture, at least partially enclosing the core region (a),

c) preferably a powdering from a powdered solvent at least partially surrounding the shell (b) on the side opposite the core.

The invention concerns an oxygen-absorbing, free-flowing granulate, a method for production of such a granulate, as well as its use for absorption of oxygen from the gas phase.

Numerous oxygen absorbents are known in the prior art. U.S. Pat. No. 2,758,932 describes a dispersion of water, glucose and an enzyme mixture with glucose oxidase and catalase activity. The dispersion is separated from the product by a water-resistant, oxygen-permeable barrier/layer. The dispersions are liquid or gel-like preparations.

DE-OS 25 20 792 describes an oxidation protective agent for beverages, in which a homogeneous mixture of glucose oxidase and catalase, as well as an inert absorbent and glucose, are added to the beverage.

U.S. Pat. No. 4,995,062 describes compositions with glucose oxidase, catalase and glucose that are added to the food, optionally in a separate sack.

EP-A-0 417 793 describes a method for packing of solid foods, as well as the package itself. The reaction between the components glucose and glucose oxidase is utilized in this case, the first component being sprayed into the package or onto a separate mechanically stable support, and the second component only being added right before packing.

EP-B-0 595 800 describes a packaging material for removal of oxygen from packaging and a method for production of this material. The packaging material according to the invention consists of two sheets, between which a liquid oxygen-absorbing formulation is sealed. The oxygen-absorbing formulation contains an enzyme that causes an oxygen-consuming reaction, as well as a corresponding enzyme substrate.

DE-38 28 513, 39 02 921, as well as EP-B-0 359 925 and U.S. Pat. No. 5,028,578, describe an oxygen absorbent based on a combination of porous precipitated silica, water and a mixture of glucose and glucose oxidase, in which at least a large part of the pore intermediate spaces of the precipitated silicon and other admixed solids are kept anhydrous. The mixtures are obtained by intensive mixing of the components.

The oxygen absorbents known in the prior art, however, are either unsuitable for absorption of oxygen from the gas phase, are not present in the dry, easily handled form and/or are in need of improvement with reference to the oxygen capacity and oxygen absorption kinetics.

The object of the present invention was therefore to provide an oxygen absorbent that is presenting an easily handled form, is suitable for absorption of oxygen in the gas phase and has high and long-lasting oxygen absorption at high oxygen capacity.

This task is solved by a free-flowing oxygen-absorbing granulate according to claim 1.

Advantageous variants are mentioned in the dependent claims.

The oxygen absorbents according to the invention are present in the form of a two- or multilayered free-flowing granulate.

It was surprisingly found that the granulates so constructed, in the first place, exhibit very easy handling and proportioning and, because of the arrangement of the individual components in different adjacent layers or regions, they have high and long-lasting oxygen absorption and high oxygen capacity. The granulates according to the invention are particularly suitable for absorption of oxygen from the gas phase or from gaseous media.

The granulate according to the invention has a core region with a substrate for an oxygen-removing enzyme, as well as optionally one or more binders.

Oxygen-removing enzyme is then understood to mean an enzyme that catalyses an oxygen-consuming reaction. According to a preferred variant of the invention, it is an oxidase that oxidizes the enzyme substrate with consumption of molecular oxygen. Glucose oxidase is particularly preferred in the context of the present invention. Appropriate enzymes are familiar to one skilled in the art and are commercially available. Generally, between 10¹ and 10³ units (U) of oxygen-removing enzyme per g of substrate are used, for example, in the form of a solution of enzyme with 1000 to 4000 U/mL or a freeze-dried enzyme or enzyme mixture.

In the simplest case, the core region consists merely of a substrate for the oxygen-removing enzyme, for example, glucose. According to one variant of the invention, glucose can be used here with an appropriate particle size on the order of 0.1 to 10 mm, especially 0.2 to 5 mm. As an alternative, appropriate granulate particles can be produced by addition of water or an aqueous glucose solution to glucose powder.

The substrate for the oxygen-removing enzyme is preferably used in the form preferred by the enzyme.

If the oxygen-removing enzyme is glucose oxidase, the substrate is preferably used as glucose or glucose monohydrate.

According to a preferred variant, the core region of the granulates according to the invention is produced according to a granulation or agglomeration method, which will be taken up further below. However, it is also possible to produce the core region in the context of ordinary compaction or extrusion. In many cases, it is then preferred that one or more binders or granulation auxiliaries be added to the substrate of the oxygen-removing enzyme for easier granulation or stronger cohesion. Such binders are familiar to one skilled in the art.

According to a preferred variant of the invention, the binders are powdered solids. Powdered solids with a particle size (d₅₀)<about 100 μm can be used, in particular. Appropriate binders, without restricting the invention to them, includes zeolite, precipitated silica, alkali or alkaline earth carbonates, citrates and phosphates, hydrotalcite, microcrystalline cellulose, phyllosilicates and/or activated carbons.

Binders in the context of the present invention, however, can also be understood to mean a liquid or liquid composition, for example, the aforementioned aqueous glucose solution or a PDA solution.

According to a particularly preferred variant, the core region of the granulates according to the invention primarily consist of, i.e., more than 50 wt. %, the substrate of an oxygen-removing enzyme. Ordinary binders can be used advantageously with 1 to 20 wt. % or more.

According to the invention, a shell is connected to the core region, which contains an enzyme or enzyme mixture applied to a support. This shell can fully or partially enclose the core region, in most cases the former being preferred. According to one variant of the invention, the shell can also enclose several core regions.

The enzyme used in the context of the present invention is an oxygen-removing enzyme, as just defined, or an enzyme mixture that contains additional enzymatic components, in addition to such an enzyme.

According to a particularly preferred variant of the invention, a catalase is used, in addition to glucose oxidase. Hydrogen peroxide is formed during oxidation of glucose to gluconic acid, so that the enzyme can be deactivated. Oxidation and deactivation of the enzyme are prevented by the presence of catalase and the same absorption performance can be achieved with much less (expensive) enzymes. Generally, between 5 and 500 units (U) catalase are used, for example, in the form of a solution of enzyme with 1000 to 4000 U/mL and in the form of a freeze-dried enzyme.

The powdered solids just defined can be used as support in the context of the present invention. It was found that particularly advantageous results are obtained with support that have a pH, measured in a 1% aqueous suspension, in the range from about 3 to 9, preferably from about 4 to 7. The pH value of the support is preferably adjusted to the employed enzyme or enzyme mixture, so that optimal enzyme activity is set.

It is also found that a powdered solid with a relatively high water absorption capacity can be used as support in the shell of the granulates according to the invention, especially precipitated silica, microcrystalline cellulose or starch, but also a phyllosilicate or their mixtures. Relatively high water absorption capacity is understood to mean the absorption capacity of at least 100%, as defined below. The stability of the granulate particles (very small dust fraction), as well as the flowability of the granulate, are obviously improved by the use of such materials.

Owing to the simultaneous presence of the enzyme or enzymes and at least one powdered solid with a relatively high water absorption capacity in the shell or in the shell region, good conditions are also present, therefore high enzyme activity.

In comparison with a homogeneous mixture of the individual components, smaller amounts of the water-absorbent solid (enzyme carrier), as well as a lower water content, referred to the granulate, are therefore required, in order to achieve good enzyme activity. The structure according to the invention also permits significant separation of the enzyme and substrate. In the structure of the granulates according to the invention, a core region therefore makes a higher content of substrate available for the oxygen-removing enzyme.

According to a particularly preferred variant, the granulates according to the invention also include a powdering of a powdered solid that is applied at least partially to the shell of the granulates. It was found that such powdering can significantly improve the condition and activity of the granulates, in which the powdered solids just defined according to the invention can be used.

According to another preferred variant of the invention, a powdered solid or solid mixture with high whiteness is used for powdering. It is also preferred that the powdered solid used for powdering additionally fulfills the function of a neutralization agent and/or a carbon dioxide-liberating agent.

Thus, it was found that particularly advantageous oxygen absorbents are obtained, if a material that traps or neutralizes liberated acid (gluconic acid during use of glucose/glucose oxidase) during enzymatic conversion in the adjacent shell of the granulate is used for powdering.

According to a particularly preferred variant, the function of a carbon dioxide developer is also furnished by the powdering, so that, with the choice of appropriate molar ratios, the molar amount of absorbed oxygen corresponds to the molar amount of liberated carbon dioxide and the gas volume remains unchanged during oxygen absorption.

According to a simplified method, granulates according to the invention can also be prepared, in which the powdered solids are not applied in a separate powdering to increase the whiteness, acid neutralization and/or carbon dioxide development, but are included or introduced directly into the composition of the shell.

A powdering containing calcium carbonate that can simultaneously serve for neutralization of the enzymatic regenerated acid, and also for liberation of carbon dioxide, has been shown to be particularly practicable. In the case of a glucose/glucose oxidase substrate/enzyme system, glucose and calcium carbonate are used in a molar ratio of about 1:1 to 3:1, especially about 2:1. Calcium carbonate also offers the advantage of high whiteness, as is desired, for example, in pharmaceutical applications of the granulate. According to one variant, an enzyme solution can also be sprayed directly onto the substrate-containing core region and a shell of calcium carbonate then applied.

It was surprisingly found that the two- or three-layered granulates according to the invention are very free-flowing and represent almost dust-free mixtures, in contrast to homogeneous mixtures known in the prior art, and exhibit high and long-lasting oxygen absorption. In contrast to homogeneous mixtures, the hazard that most of the glucose oxidase reacts with the glucose (and oxygen) already during preparation of the mixture and, in so doing, reduces the oxygen absorption capacity of the finished mixture is also reduced. The advantages of the non-homogeneous structure of the granulates according to the invention, i.e., the essential or exclusive presence of a substrate in the core region and the essential or exclusive presence of a the oxygen-removing enzyme or enzyme mixture outside of the core region, especially in the shell or in the shell region, were already discussed above. Because of this, subsequent activation of the granulates by spraying or incorporation of the enzyme or enzyme mixture on the otherwise finished granulate is made possible. Essential presence of the substrate or enzyme in the aforementioned regions of the granulate is understood to mean more than 50%, preferably more than 70%, especially more than 80%, and with particular preference, more than 90%.

The structure of granulates according to the invention also permits high enzyme activity and good substrate availability. Owing to the preferred powdering, neutralization of acid generated by the enzymatic conversion, as well as the aforementioned additional functions, are also made possible.

In the context of the present invention, especially during preparation of the granulates according to the invention, a certain blending in the boundary region between the core region and shell and between the shell and powdering can occur. Such blending is acceptable in the context of the present invention, and it was found that such a transition, in many cases, can even favor the “communication” between the substrate-containing core region and the enzyme-containing shell, or between the shell and the additional functional substances contained in the powdering. However, the granulates according to the invention do not exhibit a homogeneous distribution of the individual components.

According to another aspect, the invention concerns a method for production of the oxygen-absorbing, free-flowing granulates or particles just defined. The process includes the following steps:

a) Production of a core region, containing a substrate for an oxygen-removing enzyme, as well as optionally at least a binder,

b) Application of a shell or shell layer, containing an oxygen-removing enzyme or enzyme mixture, in which the enzyme or enzyme mixture is used with or without support.

However, in many cases, it can be advantageous to apply an enzyme or enzyme mixture already immobilized on a carrier directly to the core region.

Powdering with a particle so obtained with at least one powdered solid preferably then follows, if the latter is not directly included in the composition of the shell of the granulate.

The substrates, enzymes, binders, supports or powdered solvents used in the context of the method according to the invention were defined above.

As already mentioned, an apparatus ordinarily used for granulation, agglomeration, compaction or extraction can be used to product the granulates or particles.

The process according to the invention offers high flexibility, since substrate-containing cores are produced beforehand and can be stored in stable form for a long time (without the enzyme). In addition, during preparation, as long as the temperature-sensitive enzyme or enzyme mixture has still not been applied in the shell or shell layer, elevated temperatures can be used, for example, temperature control during agglomeration, compaction or extrusion is superfluous, or the method can be accelerated at elevated temperature. As mentioned above, subsequent activation by addition or introduction of the enzyme is also possible.

Another aspect according to the invention concerns the use of the granulate just defined or a granulate produced according to the method just discussed for absorption of oxygen, especially from the gas phase.

Such oxygen absorption agents are used in numerous applications to remove oxygen from packaging, for example, packaging of oxygen-sensitive pharmaceutical, electronic or chemical products, as well as foods.

Oxygen absorbers are finding increasing application in the packaging of pharmaceutical products. Since a number of active ingredients or drugs are oxygen-sensitive, an attempt has traditionally been made to develop an oxygen-insensitive form of administration. The development of such a formulation is very time- and cost-intensive and not practicable with the now common short “time-to-market” times. Either the use of oxygen-sensitive forms of administration in a cost-intensive oxygen barrier package or the choice of a more cost-effective packaging concept, together with an oxygen absorber, offer a way out.

The granulates according to the invention can be used in any proportions. The granulates according to the invention are preferably added in a flexible or shape-stable container to the oxygen-sensitive product or its package. According to a particularly preferred variant, a sack or canister with gas-permeable walls that contains the oxygen-absorbing granulate according to the invention is included in the product or its package.

When used in pharmaceutical packages, it is particularly advantageous if the absorption agent is added in a shape-stable container (so-called canister) to the package. These containers are made from hard plastic and, in many cases, permit the absorption agent to be added more reliably and quickly to the pharmaceutical package than would be possible with a flexible sack. Special insertion machines for canisters in pharmaceutical plastic bottles reach speeds of up to 800 per minute, whereas the insertion of sacks must generally occur manually or with machines with speeds up to 150 per minute.

Since these canisters have very much larger holes for air exchange with their surroundings, because of their design, it is particularly important for this application to use a dust-free and readily free-flowing granulate of the absorption agent, as furnished according to the invention.

The invention is now further explained by means of the following non-restrictive examples:

### EXAMPLES

### Example 1

### (Comparison)

7.5 g glucose (Merck KgaA, Germany), 1.9 g calcium carbonate (Merck KgaA, Germany) and 2.0 g microcrystalline cellulose (Vivapur Type 12, J. Rettenmaier & Söhne GmbH, Germany) were intimately mixed and sealed in a barrier sack. The barrier sack was produced from a composite PET/AL/PE foil with an edge length of 30 cm and provided with a gas valve and a septum. The closed sack was evacuated via the valve and then filled with 2 L air. 2.0 g of a solution containing 375 U glucose oxidase (OxyGo 1500, Genencor International Inc., Holland) and water were sprayed into the sack with an injector through the septum. The composition of the sample is summarized in Table 1. The oxygen concentration in the barrier sack was determined by means of a gas chromatograph. Samples were taken after 6, 24 and 72 hours. The oxygen concentrations are summarized in Table 2 as a function of time.

### Example 2

2000 g glucose (Roferose S T, Roquett GmbH, Germany) and 500 g microcrystalline cellulose (Vivapur Type 12. J. Rettenmaier & Söhne GmbH, Germany) were introduced to a mixing unit of the R02 type from Maschinenfabrik Gustav Eirich and mixed at 3000 rpm. After 5 minutes, 363 g water was sprayed in and agitated until a granulate was formed. The finished granulate was sprayed during agitation (mixing speed 900 rpm) with 67 g of solution containing 100,000 U glucose oxidase (OxyGo 1500, Genencor International Inc., Holland) and water. 500 g calcium carbonate (Merck KgaA, Germany) was then added to the granulate and mixed until the calcium carbonate was fully absorbed. The composition of the sample is summarized in Table 1. 12.9 g of the oxygen-absorbing granulate was sealed in a barrier sack. The closed sack was evacuated via the valve and then filled with 2 L of air. The oxygen concentration in the barrier sack was determined by means of a gas chromatograph. Samples were taken after 6, 24 and 72 hours. The oxygen concentration is summarized in Table 2 as a function of time.

### Example 3

2000 g glucose (Roferose S T, Roquett GmbH, Germany) and 500 g microcrystalline cellulose (Vivapur Type 12, J. Rettenmaier & Söhne GmbH, Germany) were introduced to a mixing unit of the R02 type from Maschinenfabrik Gustav Eirich and mixed at 3000 rpm. After 5 minutes, 350 g was sprayed in and agitated until a granulate was formed. The mixer was then emptied and the granulate stored in a closed sack for 5 days. 2000 g of this granulate and 200 g microcrystalline cellulose (Vivapur Type 12, J. Rettenmaier & Söhne GmbH, Germany) were introduced to the mixing unit. During agitation (mixing speed 900 rpm), 280 g of a solution containing 70,200 U glucose oxidase (OxyGo 1500, Genencor International Inc., Holland) and water were sprayed, during which a shell was formed around the granulate cores. 351 calcium carbonate (Merck KgaA, Germany) was then added to the formed core-shell granulate and mixed until the calcium carbonate was fully bonded to the granulate surface. The composition of the sample is summarized in Table 1. 15.1 g of the oxygen-absorbing granulate was sealed into a barrier sack. The closed sack was evacuated via the valve and then filled with 2 L of air. The oxygen concentration in the barrier sack was determined with a gas chromatogram. Samples were taken after 6, 24 and 72 hours. The oxygen concentration is shown in Table 2 as a function of time.

### Example 4

2000 g glucose (Roferose S T, Roquett GmbH, Germany) and 400 g microcrystalline cellulose (Vivapur Type 12. J. Rettenmaier and Söhne GmbH, Germany) were intimately mixed. The powder mixture was compacted in a compactor of the type L200/50P fro the Hutt company. The compactate was subjected to size reduction in a laboratory mortar and screened through a laboratory sieve with a mesh width of 0.5 mm and 1.0 mm. 9.0 g of the core granulate with a diameter of 0.5<d<1.0 mm was sprayed with 2.0 g of a solution containing 375 U glucose oxidase (OxyGo 1500, Genencor International Inc., Holland) and water. 1.9 g calcium carbonate was then added and mixed with a spatula until the calcium carbonate was fully absorbed. The composition of the sample is summarized in Table 1. The obtained oxygen-absorbing granulate was sealed into a barrier sack. The closed sack was evacuated via the valve and then filled with 2 L of air. The oxygen concentration in the barrier sack was determined with a gas chromatograph. Samples were taken after 6, 24 and 72 hours. The oxygen concentrations are shown in Table 2 as a function of time.

TABLE 1

| | | Calcium | Microcrystalline | Glucose oxidase | |
|---|---|---|---|---|---|
| Example | Glucose | carbonate (%) | cellulose (%) | 1500 U/mL (%) | Water (%) |
| 1 | 55.97 | 14.18 | 14.93 | 1.87 | 13.06 |
| 2 | 58.31 | 14.58 | 14.58 | 1.94 | 10.59 |
| 3 | 49.58 | 12.40 | 19.46 | 1.65 | 16.91 |
| 4 | 58.14 | 14.73 | 11.63 | 1.94 | 13.56 |

TABLE 2

| | |
|---|---|
| Oxygen | Time |

| concentration | 0 | 6 | 24 | 72 |
|---|---|---|---|---|
| Example | [h] | [h] | [h] | [h] |
| 1 | 20.9 | 11.1 | 6.6 | 5.2 |
| 2 | 20.9 | 15.7 | 3.7 | 1.5 |
| 3 | 20.9 | 15.1 | 2.4 | 0.7 |
| 4 | 20.9 | 12.9 | 4.7 | 2.8 |

### Example 5

2000 g glucose (Roferose S T, Roquett GmbH, Germany) and 500 g precipitated silica (Sipernat 22, Degussa AG, Germany) were introduced to a mixing unit of the R02 type from Maschinenfabrik Gustav Eirich and mixed at 3000 rpm. After 5 minutes, 400 g water was sprayed in and agitated until a granulate had formed. The mixer was emptied and the granulate was stored in a closed container for 4 days. 200 g precipitated silica (Sipernat 22, Degussa AG, Germany) was introduced to the mixing unit. During agitation (mixing speed 900 rpm), 360 g of a solution containing 69,000 U glucose oxidase (OxyGo 1500, Genencor International Inc., Holland) and water were sprayed. 2000 g of the granulate was then added during agitation, in which a shell was formed around the granulate cores. 345 g calcium carbonate (Merck KgaA, Germany) was then added to the formed core-shell granulate and mixed until the calcium carbonate was fully bonded to the granulate surface. The composition of the sample is summarized in Table 3. 15.8 g of the oxygen-absorbing granulate was sealed into a barrier sack. The closed sack was evacuated via the valve and then filled with 2 L of air. The oxygen concentration in the barrier sack was determined with a gas chromatograph. Samples were taken after 6, 24 and 72 hours. The oxygen concentrations are shown in Table 4 as a function of time.

TABLE 3

| | | Calcium | | | |
|---|---|---|---|---|---|
| Ex- | | carbonate | Precipitated | Glucose oxidase | Water |
| ample | Glucose | (%) | silica (%) | 1500 U/mL (%) | (%) |
| 5 | 47.47 | 11.88 | 18.76 | 1.58 | 20.31 |

TABLE 4

| | |
|---|---|
| Oxygen | Time |

| concentration | 0 | 6 | 24 | 72 |
|---|---|---|---|---|
| Example | [h] | [h] | [h] | [h] |
| 5 | 20.9 | 15.3 | 2.7 | 0.8 |

### Example 6

2000 g glucose (Roferose S T, Roquett GmbH, Germany) and 500 g precipitated silica (Sipernat 22. Degussa AG, Germany) were introduced to a mixing unit of the R02 type from Maschinenfabrik Gustav Eirich and mixed at 3000 rpm. After 5 minutes, 400 g water was sprayed in and agitated until a granulate was formed. The mixer was emptied and the granulate was stored in a closed vessel. 2000 g of this granulate and 200 g microcrystalline cellulose (Vivapur Type 12, J. Rettenmaier & Söhne GmbH, Germany) were introduced to the mixing unit. During agitation (mixing speed 900 rpm), 280 g of a solution containing 69,000 U glucose oxidase (OxyGo 1 500, Genencor International Inc., Holland) and water were sprayed on, during which a shell was formed around the granulate cores. 345 g calcium carbonate (Merck KgaA, Germany) was then added to the formed core-shell granulate and mixed until the calcium carbonate was fully bonded to the granulate surface. The composition of the sample is summarized in Table 5. 15.4 g of the oxygen-absorbing granulate was sealed into a barrier sack. The closed sack was evacuated via the valve and then filled with 2 L of air. The oxygen concentration in the barrier sack was determined with a gas chromatograph. Samples were taken after 6, 24 and 72 hours. The oxygen concentrations are shown in Table 6 as a function of time.

### Example 7

2000 g glucose (Roferose S T, Roquett GmbH, Germany) and 500 g bentonite (Printosil, Süd-Chemie AG, Germany) were mixed in a mixing unit of the R02 type from Maschinenfabrik Gustav Eirich at 3000 rpm. After 5 minutes, 175 g water was sprayed in and agitated until a granulate was formed. The mixer was emptied and the granulate was stored in a closed vessel. 2000 g of this granulate and 200 g microcrystalline cellulose (Vivapur Type 12, J. Rettenmaier & Söhne, GmbH, Germany) were introduced to the mixing unit. During agitation (mixing speed 900 rpm), 280 g of a solution containing 75,750 U glucose oxidase (OxyGo 1500, Genencor International Inc., Holland) and water were sprayed on, during which a shell was formed around the granulate cores. 374 g calcium carbonate (Merck KgaA, Germany) was then added to the formed core-shell granulate and mixed until the calcium carbonate was fully bonded to the granulate surface. The composition of the sample is summarized in Table 5. 14.3 g of the oxygen-absorbing granulate was sealed into a barrier sack. The closed sack was evacuated via the valve and then filled with 2 L of air. The oxygen concentration in the barrier sack was determined with a gas chromatograph. Samples were taken after 6, 24 and 72 hours. The oxygen concentrations are shown in Table 6 as a function of time.

### Example 8

2000 g glucose (Roferose S T, Roquett GmbH, Germany) and 500 g zeolite (Wessalit P, Degussa AG, Germany) were introduced to a mixing unit of the R02 type from Maschinenfabrik Gustav Eirich and mixed at 3000 rpm. After 5 minutes, 200 g water was sprayed in and agitated until a granulate had formed. The mixer was emptied and the granulate stored in a closed vessel. 2000 g of this granulate and 200 g microcrystalline cellulose (Vivapur Type 12. J. Rettenmaier & Söhne, GmbH, Germany) were introduced to the mixing unit. During agitation (mixing speed 900 rpm), 280 g of a solution containing 74,050 U glucose oxidase (OxyGo 1500, Genencor International Inc., Holland) and water were sprayed on, during which a shell was formed around the granulate cores. 370 g calcium carbonate (Merck KgaA, Germany) was then added to the formed core-shell granulate and mixed until the calcium carbonate was fully bonded to the granulate surface. The composition of the sample is shown in Table 5. 14.4 g of the oxygen-absorbing granulate was sealed in a barrier sack. The closed sack was evacuated via the valve and then filled with 2 L of air. The oxygen concentration in the barrier sack was determined with a gas chromatograph. Samples were taken after 6, 24 and 72 hours. The oxygen concentrations are shown in Table 6 as a function of time.

TABLE 5

| | | | | | Glucose | |
|---|---|---|---|---|---|---|
| | | Calcium | | Microcrystalline | oxidase 1500 | |
| Example | Glucose | carbonate (%) | Binder (%) | cellulose (%) | U/mL (%) | Water (%) |
| 6 | 48.81 | 12.21 | 12.21 | 7.08 | 1.63 | 18.05 |
| 7 | 52.38 | 13.10 | 13.10 | 7.01 | 1.75 | 12.65 |
| 8 | 51.96 | 13.00 | 13.00 | 7.02 | 1.73 | 13.29 |

TABLE 6

| | |
|---|---|
| Oxygen | Time |

| concentration | 0 | 6 | 24 | 72 |
|---|---|---|---|---|
| Example | [h] | [h] | [h] | [h] |
| 6 | 20.9 | 15.5 | 2.8 | 0.9 |
| 7 | 20.9 | 16.3 | 5.4 | 2.7 |
| 8 | 20.9 | 16.5 | 6.2 | 3.1 |

### Example 9

14.4 g of the oxygen-absorbing granulate from example 6 was filled into a total of 5 canisters (cylindrical, diameter 1.9 cm, height 2.4 cm; each with 360 holes 150 μm in diameter in the cover and bottom of the cylinder). The canisters were sealed into a barrier sack. The closed sack was evacuated and then filled with 2 L of air. The oxygen concentration in the barrier sack was determined with a gas chromatograph. Samples were taken after 6, 24, 72 and 96 hours.

| 0 Hours | 6 Hours | 24 Hours | 72 Hours | 96 Hours |
|---|---|---|---|---|
| 20.9% | 17.0% | 6.3% | 3.5% | 1.1% |

Evaluation* of the granulates:

| Example | Flowability | Freedom from dust | Stability |
|---|---|---|---|
| 1 | −− | −− | −− |
| 2 | + | ++ | ++ |
| 3 | ++ | ++ | ++ |
| 4 | ∘ | ∘ | ∘ |
| 5 | ++ | + | ++ |
| 6 | ++ | ++ | ++ |
| 7 | ++ | ++ | ++ |
| 8 | ++ | ++ | ++ |

*++ very good; + good; ∘ moderate, − poor; −− very poor

Determination of Water Absorption Capacity:

Precisely 3.0 g of the material was weighed into a weighed sieve with pleated filter. The filled sieve was suspended in a beaker with water, so that the material was fully covered with water. After an absorption time of 20 minutes, the sieve was suspended for another 20 minutes in an empty beaker for dripping. After dripping, the sieve and contents were weighed again. The water absorption capacity was obtained from the ratio of the absorbed amount of water to the initial weighed amount. The determination was performed as a double determination. The difference between the two determinations was less than 10%.

| Binder | Water absorption capacity (%) |
|---|---|
| Precipitated silica (Sipernat 22, Degussa | 349 |
| AG) | |
| Microcrystalline cellulose | 298 |
| (Vivapur Type 12, JRS GmbH) | |
| Bentonite (Printosil, Sud-Chemie AG) | 349 |
| Zeolite (Wessalit P, Degussa AG) | 51 |
| Calcium carbonate (Merck KgaA) | 92 |

1. An oxygen-absorbing granulate with a non-homogenous structure, comprising
a core region, comprising a substrate for an oxygen-removing enzyme, and
a shell region, at least partially surrounding the core region, comprising the oxygen-removing enzyme.

2-23. (Canceled)

24. The oxygen-absorbing granulate of claim 1 further comprises a shell region enclosing powdered solid, at least partially enclosing the shell region.

25. The oxygen-absorbing granulate of claim 1 wherein the core region further comprises a binder.

26. The oxygen-absorbing granulate of claim 1 wherein at least 80 weight percent have a diameter from about 0.1 to about 10 mm.

27. The oxygen-absorbing granulate of claim 1 wherein the oxygen-removing enzyme comprises an oxidase.

28. The oxygen-absorbing granulate of claim 27 wherein the oxidase comprises a glucose oxidase.

29. The oxygen-absorbing granulate of claim 1 wherein the shell region further comprises a support to which the oxygen-removing enzyme is applied.

30. The oxygen-absorbing granulate of claim 25 wherein the binder comprises a binder powdered solid having a particle size (d₅₀) less than about 100 μm.

31. The oxygen-absorbing granulate of claim 25 wherein the binder comprises an aqueous liquid blended with a material selected from an aqueous glucose solution, PVA and mixtures thereof.

32. The oxygen-absorbing granulate of claim 30 wherein the binder powdered solid is selected from the group consisting of zeolite, precipitated silica, an alkali or alkaline earth carbonate, citrate or phosphate, hydrotalcite, cellulose or cellulose derivatives, layered silicates, bentonites, and other silicate support materials, activated carbons, and mixtures thereof.

33. The oxygen-absorbing granulate of claim 24 wherein the shell region enclosing powdered solid is selected from the group consisting of zeolite, precipitated silica, an alkali or alkaline earth carbonate, citrate or phosphate, hydrotalcite, cellulose or cellulose derivatives, layered silicates, bentonites, and other silicate support materials, activated carbons, and mixtures thereof.

34. The oxygen-absorbing granulate of claim 29 wherein the support has a pH value in the range from about 3 to about 9.

35. The oxygen absorbing granulate of claim 29 wherein the support comprises a support powdered material selected from the group consisting of zeolite, precipitated silica, an alkali or alkaline earth carbonate, citrate or phosphate, hydrotalcite, cellulose or cellulose derivatives, layered silicates, bentonites, and other silicate support materials, activated carbons, and mixtures thereof.

36. The oxygen-absorbing granulate of claim 35 wherein the support powdered solid has an liquid-absorption capacity of at least about 100 percent.

37. The oxygen-absorbing granulate of claim 24 wherein the shell region enclosing powdered solid has a high whiteness value.

38. The oxygen-absorbing granulate of claim 24 wherein the shell region enclosing powdered solid comprises a neutralization agent.

39. The oxygen-absorbing granulate of claim 24 wherein the shell region enclosing powdered solid liberates CO₂ and/or another gas during acidification.

40. The oxygen-absorbing granulate of claim 1 wherein the substrate comprises glucose and/or glucose monohydrate.

41. The oxygen-absorbing granulate of claim 1 wherein the shell region at least partially encloses at least two core regions.

42. A method for production of an oxygen-absorbing granulate comprising:
producing a core region comprising a substrate for an oxygen-removing enzyme, and
generating a shell region containing the oxygen removing enzyme at least partially surrounding the core region to form the granulate.

43. The method of claim 42 further comprising powdering the oxygen-absorbing granulate with at least one shell region enclosing powdered solid.

44. The method of claim 42 further comprising adding at least one binder to the core region.

45. The method according to claim 42 further comprising producing the shell region from an outer layer of the core region by application of the enzyme to the core region.

46. A process for the absorption of oxygen by placing the granulate material of claim 1 within a container containing a gaseous environment comprising oxygen.

47. The process of claim 46 wherein the container comprises a storage unit further containing electronic or pharmaceutical products.

48. The process of claim 46 wherein the granulate material is placed within a sac, canister or the like containing at least one gas permeable wall prior to placement within the container.
